(19) United States
(12) Patent Application Publication
(10) Pub. No.: US 2011/0308985 A1
(43) Pub. Date: Dec. 22, 2011

(54) COMPOSITION OF A LIPOSOMAL GEL CONTAINING HYDROCORTISONE, ITS METABOLITES, PRECURSORS OR MIXTURES THEREOF AND THE USE THEREOF

(76) Inventor: Gina Van Bogaert, Hoeilaart (BE)

(21) Appl. No.: 13/203,059

(22) PCT Filed: Feb. 26, 2010
(86) PCT No.: PCT/BE2010/000015

§ 371 Date: Aug. 24, 2011

(30) Foreign Application Priority Data
Feb. 26, 2009 (BE) 2009/0120

(51) Int. Cl. B65D 85/00; A61K 31/573; A61P 5/44; A61K 9/127

(52) U.S. Cl. 206/459.1; 424/450; 514/179

(57) ABSTRACT

Liposomal gel which, apart from liposomes and a continuous phase, includes at least one thickening agent, at least one biologically active material and, optionally, at least one additive, whereby the biologically active material is encapsulated in the liposomes, and whereby this material consists of hydrocortisone, its metabolites or precursors, or a mixture thereof, and is applied on the skin for therapeutic purposes, characterised in that the weight ratio phosphatidylcholine/hydrocortisone amounts to at least 2/1 and/or in that the weight ratio phosphatidylcholine/carbomer hydrogel amounts to at least 20/1.

The present invention concerns the composition of a liposomal gel containing hydrocortisone, its derivatives, metabolites, prodrugs or mixtures thereof and the use thereof.

In particular, the invention aims a liposomal gel containing pharmaceutical compounds which comprise hydrocortisone, its metabolites or precursors, or mixtures thereof and which are applied on the skin in an active dose, among others but not exclusively with the aim to maintain or restore the hormonal balance in the body, in other words to be used in HRT (Hormonal Replacement Therapy) with applications in endocrinology and other specialties.

By hydrocortisone is understood in this context the primary steroid belonging to the category of the corticosteroids and according to the following chemical formula:
pregn-4-ene-3,20-dione,11,17,21-trihydroxy-,(11beta)-, or
11,17,21-trihydroxy-,(11beta)-pregn-4-ene-3,20-dione,
or (11beta)-11,17,21-trihydroxypregn-4-ene-3,20-dione,
also known by the trivial names of Cortisol; 4-pregnene-11beta,17alpha,21-triol-3,20-dione; 17-hydrocorticosterone; anti-inflammatory hormone; Kendall's compound F; Reichstein's substance M; etc.

The molecular formula of hydrocortisone is formed of 21 carbon atoms, 30 hydrogen atoms and 5 oxygen atoms, and the Chemical Abstract Service (CAS) registration number is 50-23-7.

By the name metabolites of hydrocortisone is also understood in this context among others tetrahydrocortisone, or 3,17-dihydroxy-17-(2-hydroxyacetyl)-10,13-dimethyl-2,3,4,5,6,7,8,9,12,14,15,16-dodecahydro-1H-cyclopenta(a)phenatrene-11-one, formed of 21 carbon atoms, 32 hydrogen atoms, and 5 oxygen atoms, and which is better known under different synonyms.

In this context are known oral administration forms of hydrocortisone containing compounds, for example by the trade name of Hydrocortisone®, of the company Erfa S.A.

These and equivalent compounds tend to show the following inconvenient disadvantages in oral administration forms:
A very limited biological availability (only 3 to 4 hours) due to a short biological half-life of about 100 minutes. This implies that the intake must be repeated every 4 hours, which leads to bad therapy compliance and unstable serum concentrations;
The maximum plasma concentration is reached about one hour after the intake of the tablet;
High serum peaks of (supraphysiological) hydrocortisone occur about 1 hour after the intake of a tablet, which is well over 3 times higher than the physiological morning peak.
An intake of four times per day then results in 4 supraphysiological serum peaks of hydrocortisone with metabolic and endocrinologic consequences;

A response or rise of the other stress hormones, growth hormone, thyroid gland hormone, testosterone and/or oestradiol may occur as a result of the high serum levels about one hour after oral administration. In time, this leads to metabolic and hormonal disorder whereby any of the following complaints may occur, among others:
Reversible or permanent adrenal gland arrest, accompanied by the suppression of the adrenocorticotropic hormone production, especially of cortisole but also of other adrenal hormones such as testosterone, dehydroepiandrosterone and aldosterone may also occur;
Interference with the biofeedback accompanied by the suppression of ACTH (hypophysar adrenal gland gonadotropine) resulting in adrenal gland arrest is possible as well;
Moisture retention due to hypokalemia with swollen tissues and hypertension may occur.

The supraphysiological hydrocortisone serum peaks cause a rise in aldosterone—a hormone with an anti-diuretical action.

Also rises of growth hormone and oestradiol may reinforce said moisture retention.
Development of Cushing Syndrome with fat redistribution, diabetes, hypertension, insomnia, overvoltage, osteoporosis, weakening of skin and connective tissue—resulting from the supraphysiological serum peaks was sometimes recorded;
Oral administration of higher doses of hydrocortisone are necessary because of the gastrointestinal loss and first pass effect due to liver passage with a metabolisation of the molecule in either or not active metabolites;
Formation of supraphysiological concentrations of active metabolites such as tetrahydroxycortisol and tetrahydrocortisone is among the possibilities.

Compounds of bioidentical hydrocortisone which may be injected intramuscularly or intravenously are known as well, for example by the trade name Solu-Cortef® of the company Pharmacia Corporation (Pfizer).

The inconvenient disadvantages of the injectable form of hydrocortisone may be any of the following, among others:
very limited biological availability (biodisponibility);
no physiological diurnal variation of bioavailability possible, meaning no application in HRT—application in anaesthetics;
extremely high supraphysiological serum concentrations;
extreme hormonal stress response, among others of growth hormone, testosterone, oestradiol, thyroid gland hormone;
with massive metabolisation of hydrocortisone in either or not active metabolites;
temporary to permanent adrenal gland suppression.

Instead of the injections with hydrocortisone as mentioned above, it is also known to use a replacement therapy in the case of adrenal cortex insufficiency of the zona fasciculata, with insufficient hydrocortisone production, based on injectable synthetic derivatives of mineralocorticoides, either or not with depot action.

Known preparations in this context are any of the following, among others:

1. Methylprednisolone, by the following trade names:

### Methylprednisolone® (Faulding)

### Solu-Medrol® (Pharmacia)

### Depo-Medrol® (Pharmacia);

2. Betamethasone, having as typical trade names:

### Celstone® (Shering-Plough)

### Diprophos® (Shering-Plough);

3. Dexamethasone, having as trade name:

### Oradexone® (Organon);

4. Prednisolone, known by the trade names:

### Deltacortril® (Pfizer)

### Prednicort® (Continental Pharma)

### Prednicortelone® (Continental Pharma)

### Solu-Dacortine® (Merck);

### 5. Triamcinolone;

### 6. Budenonide.

The inconvenient disadvantages of the injectable form of these synthetic derivatives of mineraloglucocorticoides as a replacement of hydrocortisone may be any of the following, among others:
They are not available as a bioidentical molecule;
They have no substitute value, they have no application as a substitute in case of adrenal cortex insufficiency, and they can only be applied in emergency medicine and orthopaedics;
They only have an anti-inflammatory effect;
There is no physiological diurnal release of the product.

Indeed, these products on average release their molecule during 3 weeks, resulting in very high serum concentrations during the week following the injection, followed by a slow, reduced availability up to three weeks after the injection;
The oil suspension can only contain maximally up to 10% of the active product;
The product may crystallize out intramuscularly, with a decreasing depot action as a direct result.

A further known disadvantage of the oral administration form as well as the intramuscularly or intravenously injectable forms is that they are all based on substitution with supraphysiological doses of hydrocortisone, increasing the risk of system effects occurring, such as among others:
Water and electrolyte disorders: hypokalemic alkalosis, water/salt retention, sometimes accompanied by arterial hypertension or even congestive heart insufficiency;
Endocrinic and metabolic disorders:
   Iatrogenic Cushing's syndrome, reversible reduced glucose tolerance, reversible diabetes, growth arrest in children, irregular menses;
Muscle and bone disorders:
   Muscle weakness and muscle atrophy, osteoporosis, aseptic necrosis of the hip ball;
Gastrointestinal disorders:
   Gastric and duodenal ulcers with possible haemorrhages and perforation;
   Acute pancreatitis;
Skin diseases:
   Acne, hypertrichosis, ecchymoses, purpura, decelerated wound healing;
Adrenal gland arrest:
   Reversible but sometimes also permanent;
Eye disorders:
   Cataract and glaucoma.

The use of compounds containing hydrocortisone and which are applied on the skin as a topical or locally, for example in the shape of a cream, balm or ointment is known as well.

In this connection can be mentioned for example the 1% hydrocortisone ointment Cremicort® of the company Omega Pharma or ditto cream Cortril® of the company Pfizer.

These ointments or creams, according to the official publication “Hydrocortisone Official FDA Information, side effects and uses”, are characterised among others by anti-inflammatory, antipruritical and vasoconstringent qualities, and they are also used to that end, especially for the treatment of certain skin disorders.

The penetration capacity of the active components through the skin is restricted, however, for several reasons.

Factors such as among others the intactness of the epidermal barrier play a major role here, sometimes requiring occlusive dressings or the ointment or cream having to be applied over larger skin surfaces in order to obtain the required therapeutic effect, especially in the case of resistant forms of skin disorders.

The restricted penetration capacity of a typical hydrocortisone ointment or cream through the skin also implies that such a cream cannot be used for any other purposes than the local treatment of skin disorders.

Such an ointment or cream, for example Anusol-HC 2.5% ointment, is typically composed as follows:

Hydrocortisone, active ingredient, 2.5%, processed in inactive ointment-forming components such as benzyl alcohol, petrolatum, stearyl alcohol, propylene glycol, isopropyl myristate, polyoxyl 40 stearate, carbomer 934 (thickening agent), sodium lauryl sulphate, EDTA disodium salt (chelating agent), sodium hydroxide (neutralizer and pH adjustor) and water.

A typical hydrocortisone cream, for example formula No. 0321/1326 of Actavis Mid Atlantic LLC, is composed for example of:

Hydrocortisone, active ingredient, 10 mg in 1 gram cream, further also stearyl alcohol, glyceryl monostearate, polyoxy 40 stearate, isopropylpalmitate, paraffin, sorbitan monostearate, glycerine, lactic acid, potassium sorbate and water.

Inconvenient disadvantages of local forms may be among others any of the following ones:
they have no system resorption, unless in case of long-term use of high concentrations of ointments under occlusive dressings;
they only have dermatological applications;
and they have high local effects, with typical local side effects resulting from long-term local applications such as:
irreversible atrophy with connective tissue weakness, striae, bruises, haematomae, haemorrhages;
causing ulcers or wounds, and disturbed wound healing;
they may promote secondary infection of the skin and/or wounds.

Finally, the local corticoids do not offer any causal treatment, as the complaint returns as soon as the treatment is ended.

Apart from the administration of hydrocortisone in the form of an ointment or cream, also the administration of hydrocortisone in the form of a liposomal gel is examined.

Thus, Kim et al. (Journ. Of Controlled Release 46 (1997), 243-251) compared the percutaneous absorption in mice of hydrocortisone applied in the form of a liposomal gel or in the form of an ointment between normal skin and skin of which the epidermis (stratum corneum) had been removed. The percutaneous absorption of hydrocortisone through the skin without any epidermis was significantly faster than in the skin with an epidermis for the liposomal gel, which indicates that the epidermis functions as a barrier for the liposome-linked hydrocortisone.

According to this study, the liposomal gel reduces the absorption of hydrocortisone in the skin compared to conventional ointment formulas, but despite said reduced absorption, higher concentrations of hydrocortisone were obtained in the skin with the liposomal gel; they could moreover be maintained longer than in the case of ointments.

According to said researchers, the delayed diffusion of hydrocortisone from the skin to the systemic blood circulation appears to be a potential factor in maintaining the hydrocortisone concentrations from the liposomal gel in the skin.

The administration of hydrocortisone in the form of a liposomal gel as described is still problematic in that the amount of hydrocortisone which is absorbed through the skin and ends up in the blood circulation is insufficient to mimic a natural diurnal cycle of plasma concentrations in the blood.

This is illustrated in FIG. 4 of the above-mentioned reference of Kim et al., in which the concentration of hydrocortisone in plasma as a function of time is compared between hydrocortisone administered as a liposomal gel and as an ointment.

The invention aims to provide a compound for applying a pharmaceutically active concentration on the skin in a dosage form containing hydrocortisone, its metabolites or precursors, or mixtures thereof as active ingredient(s), and which remedies the above-mentioned and other disadvantages, in particular the disadvantages related to the oral form, the topical, the injectable intramuscular (depot) preparations, or the intravenously injectable preparations, or the liposomal gel compounds which are inadequate.

The invention further aims to provide a compound containing hydrocortisone, its metabolites, its precursors or mixtures thereof in such a galenic form that, when applied on healthy skin in an effective concentration, it guarantees such a permeability of the active component through the healthy skin that the compound can be used without any noticeable side effects, among others but not exclusively in Hormonal Replacement Therapy (HRT), in endocrinology or other specialties.

The invention further aims to provide the compound containing the pharmaceutically effective hydrocortisone, its metabolites or precursors or mixtures thereof in a package which makes it possible to easily and precisely weigh or offer a predetermined dose thereof.

Said aims are reached thanks to the composition and use of a liposomal gel which, apart from liposomes and a continuous watery phase, contains at least one thickening agent, at least one biologically active material and, optionally, at least one additive, whereby the biologically active material is encapsulated in the liposomes, and whereby this material consists of hydrocortisone, its metabolites or precursors, or a mixture thereof, and is applied on the skin for therapeutic purposes.

The name “liposomes” must be understood in the broader sense within this context and also comprises liposomes of the niosome type which are built of non-ionogenic amphiphiles and contain an aliphatic, saturated, lipophilic chain on the lipophilic side.

Said aims are also reached by providing the gel in an easily dispensable form, for example by using a container equipped with a manually operated pump, or by packing it for example in soft gelatine capsules containing a specifically required pharmaceutically active dose and which can be easily opened before use.

A major advantage of a gel according to the invention is that the compound can be administered transdermally.

Another advantage is that the concentration of active ingredients, i.e. the fat-soluble bioidentical hydrocortisone molecule, its metabolites, precursors or mixtures thereof can be increased up to above 10% of the liposomal gel.

Yet another advantage is that the gel according to the invention can be used in HRT (hormonal replacement therapy), in particular as a replacement of certain conventional galenic forms, namely the oral form, the topical or local form, the intramuscularly and intravenously injectable form without the inherent disadvantages thereof.

A further advantage of the invention is that the gel can be applied without causing the classical system side effects in the long term or any long-term skin disorders on the application spot.

Another advantage of the invention is that it becomes possible to administer lower daily doses of hydrocortisone, its metabolites or precursors or mixtures thereof while obtaining normal physiological diurnal variations in the serum concentrations and with excellent compliance.

Yet another advantage is that lower doses can be used thanks to a transdermal administration to obtain the same therapeutic effect, as there is no gastrointestinal loss or first pass effect resulting from the pass through the liver with a metabolisation of the active molecule in either or not active metabolites.

A further major advantage of the invention is that the hydrocortisone liposomal gel, thanks to the specific transdermal availability of the cortisone, metabolites, precursors or mixtures thereof, is capable of mimicking the natural diurnal hydrocortisone production, together with the patient's own adrenal gland production, of for example cortisone, without producing the above-mentioned and other disadvantages.

A further advantage is that the gel according to the invention can be applied in many fields, such as for example but not exclusively in the treatment of:

### 1. Endocrine Disorders

due to primary or secondary adrenal cortex insufficiency or restricted adrenal cortex reserve;
surgical treatments, severe illness or trauma in patients with adrenal gland insufficiency or problematic adrenal cortex reserve;

### 2. Non-Endocrine Disorders

Allergic conditions
Respiratory disorders
Anaesthesia/surgery
Dermatology
Rheumatology
Intensive care
   3. Treatment of Various Diseases such as
migraine
anorexia
hypotension
pregnancy disorders caused by a low hydrocortisone serum value (vomiting, sickness, pregnancy mask, fatigue, premature contractions, . . . )
fibromyalgia,
Chronic Fatigue Syndrome, CFS.

In order to better explain the characteristics of the invention, the following preferred compositions and applications of the liposomal gel containing hydrocortisone, its metabolites, precursors or mixtures thereof according to the invention are described by way of example only without being limitative in any way.

Classical liposomes may be regarded as artificially synthesised, microscopically small follicles, usually spherical, which are formed of one or several concentric layers, also called lamellea, which consist of lipids.

The latter are compounds which have a lipophobic/hydrophilic part on the one hand and a lipophilic-hydrophobic part on the other hand.

In principle, said lamellaa are composed to a large extent or as a whole of phospholipids which, also in living creatures, form the main component of the cell membrane.

Thus, in order to form the liposome wall, phospholipids are used among others, containing mixed lipid chains and which are found in nature or which are generally selected from the group formed of lecithin, phosphatidylethanolamine, lysolecithin, lyophosphatidylethanolamine, phosphatidylserine, phosphatidylinositol, sphingomyelin, cardiolipin, phosphatidinic acid, cerebroside, stearyl amine, dipalmitoylphosphatidylcholine, phosphatylcholine, dioleolyl phosphatidylethanolamine, or any equivalent.

Inside the follicles, formed by the liposomes, can be inserted an active substance, such as in the case of the compound according to the invention for example hydrocortisone, which will then, upon application of the hydrocortisone containing liposomes on the skin, release the biologically and therapeutically active material in a controlled manner, which will finally result in a total transdermal absorption of said material.

The liposomes containing the hydrocortisone, its metabolites, precursors or mixtures thereof can be prepared according to conventional preparation methods, as described among others in Biochemica & Biophysica Acta, 475, (1976), 259-302 (authors D. A. Tyrell, T. D. Heath, C. M. Colley and B. E. Ryman.)

However, the specific method for producing the liposomes according to the invention and including the desired active component or components therein is not critical.

In a preferred embodiment, use is made of what is called a Microfluidizer® of the company Microfluidics Corp.

The working and use of such devices has been exhaustively described for example in Biochemica & Biophysica Acta, 775, 169-174 (1984) by Mayhew et alia, “Characterization of Liposomes Prepared using a Micro-emulsifier”.

In a further preferred embodiment, the liposomes containing the hydrocortisone, its metabolites, precursors or mixtures thereof are prepared by making use of ultrasonification techniques or by forming what are called reverse phase preservers, or by means of thin film hydration, or also by means of detergent dialysis.

In a further preferred embodiment, the liposomes containing the hydrocortisone, its metabolites, precursors or mixtures thereof according to the invention are first lyophilised before being hydrated again in a following step.

Finally, the liposomes containing the hydrocortisone, its metabolites, precursors or mixtures thereof are emulsified or suspended in an adapted gel.

In a preferred embodiment, they are mixed in an appropriate mixer to that end with gel matrices forming a polymeric three-dimensional structure around the liposomes, such as for example with a hydrogel consisting of neutralised carbomer, in particular of Carbopol 934® or 941.

Additives can be built in if need be, in the enclosed phase containing the hydrocortisone, its metabolites, precursors or mixtures thereof, as well as in the external gelled continuous watery phase, as in the membrane or membrane building phase, selected for example from the group formed of:

Sterols to increase the stability and permeability, skin softeners (emollients), vitamins such as for example ascorbyl palmitate or vitamin D3, nutrients, moisturizers, preservatives, radical absorbers, antioxidants, chelators such EDTA disodium salt, dyes to recognize the concentration of active ingredients, mixtures of different polyethylene glycols, poloxamers such as for example ABA block copolymers with a large variation in the polyethylene over polypropylene ratio, surfactants such as for example dodecyl sulphate, different salts, etc.

Finally, for ease of use, the liposomal gel containing the hydrocortisone, its derivatives, metabolites, precursors or mixtures thereof according to the invention is provided in a dosage unit which can offer a desired therapeutically effective volume of the gel to be applied on the skin, for example by making use of a manually operated pump system.

In another preferred embodiment, the gel according to the invention is offered and packed for example in soft gelatine capsules containing a desired therapeutically effective volume of the gel, and which are easy to break open for use.

According to a practical, preferred method, one proceeds for example as follows in the case of the application of the invention within the scope of Hormonal Replacement Therapy of hydrocortisone.

Imitation of the diurnal hydrocortisone production comes down to reaching the morning serum peak of the hydrocortisone, which maximally amounts to 25 μg/dl, whereby this serum concentration of hydrocortisone starts to decrease again about 4 hours following the application to finally drop to the patient's base level again about 15 hours following the application.

The aimed cortisoluria in a 24-hour urine collection then amounts to a urinary free cortisol level of maximally 90 μg/day and 17 —OH steroids (calculated) of maximally 35 μmol/24 hours.

Said serum values/cortisoluria, which are the result of the patient's own adrenal gland production plus the result of the bioavailability of the transdermal hydrocortisone liposomal gel applied on the skin, are brought to or maintained at the required level by using adapted therapeutic doses of the latter.

Said action was experimentally established starting with the preparation of a liposomal gel according to the invention and its application on volunteers, followed by measuring the urinary free cortisol level during a 24-hour urine collection, as will be described in the following experimental part.

### Experimental Part

The composition of three formulas of the liposomal gel is represented in table I.

TABLE I
Composition of the liposomal gel according to the invention.

| | 2% | 5% | 10% |
|---|---|---|---|
| FORMULA | (g HC) | (g HC) | (g HC) |
| Hydrocortisone (HC) | 2 | 5 | 10 |
| Phosal | 20 | 20 | 25 |
| Isopropanol | 20 | 20 | 20 |
| Almond oil | 3 | 3 | 1.5 |
| Carbomer gel 980 | 0.52 | 0.52 | 0.44 |
| Di Na-EDTA | 0.052 | 0.052 | 0.044 |
| Propylene glycol | 5.2 | 5.2 | 4.4 |
| Trometamol | 0.52 | 0.52 | 0.44 |
| Water | upto 100 | upto 100 | upto 100 |

In this composition, Phosal stands for a standardised concentrate containing at least 50% of phosphatidylcholine and between 33.8 and 41.2% propylene glycol, whereby % indicates percentage by weight.

DE carbomer gel 980 is a commercial name for a carbomer hydrogel, i.e. a synthetic polymer of high molecular weight of acrylic acid, cross-linked with allylsucrose containing 56 to 68% of carboxylic acid groups.

Trometamol is a name for 2-amino-2-hydroxymethyl-1,3-propanediol which serves as an emulsifier.

Said three compositions (2% HC, 5% HC and 10% HC) were tested on volunteers whose adrenal gland was entirely suppressed by administering bethamethasone in the form of 1 Celestone tablet of 1 mg per day, starting 3 days before the administration of the liposomal hydrocortisone gel up to the final test day included.

The suppression of the adrenal gland was verified by determining the serum cortisol level twice a day by means of a blood sample.

Next, the test formulas were applied on the skin in a dose of 5 gram, and during 24 hours following the application, the urine of the volunteers was collected to measure the urinary free cortisol (ufc) therein, which gave the following results.

TABLE II
Urinary Free Cortisol (ufc) measured after application of
liposomal gels with different hydrocortisone concentrations.

| Composition | | | | |
|---|---|---|---|---|
| liposomal gel (% HC) | 0 | 2 | 5 | 10 |
| ufc (microgram/dl) | 0 | 0.9 | 1.1 | 3.0 |
| ufc (microgram/24 h) | 0 | 12.2 | 12.1 | 28.5 |

It is preferred to measure the urinary free cortisol as a standard for the cortisone level in the bloodstream, rather than to measure the serum level of the blood itself, since the hydrocortisone is very quickly transformed in active metabolites and consequently can no longer be detected in the serum by means of the conventional method of analysis.

Since the ufc for the 2% HC and 5% HC compositions are almost equal, it may be concluded for now that the hydrocortisone volume does not determine the ufc all by itself.

However, in the 10% HC composition, the ufc increased 2.35 times, and hence also the biological availability in the volunteer.

We note that this improved result is obtained by increasing the phosphatidylcholine/hydrocortisole ratio to 2/1, and also by increasing the phosphatidylcholine/carbomer hydrogel ratio to 20/1.

Possibly, more phosphatidylcholine should be used to increase the final dimensions of the liposomes or to make them double-walled so as to be able to incorporate a lipophilic/hydrophilic molecule, what hydrocortisole is after all, in the liposomes.

The measured urinary free cortisol levels obtained by administering the 10% HC composition may be said to be satisfactory to obtain a good clinical result, whereby the natural supply of hydrocortisole by the adrenal gland can be imitated.

An advantage of such a transdermal liposomal gel treatment with hydrocortisone is that the following parameters must not be specifically followed any longer, as was the case with the oral and injectable forms. In case of the latter, they are more in particular:
the glucose level
the arterial blood pressure
the serum potassium level
infectious complications

Hereby are also applied:
Glucose and sodium restriction
Anti-tubercular prophylaxis

Any possible medicinal interactions with the above-mentioned oral and injectable forms are not neglected thereby, in particular:

### 1. Due to Hypokalemia (ECG Controlled)

Erythromycin
Certain anti-aritmics
Digitalismus
Potassium reducers

### 2. Due to Changes in the Active Plasma Levels:

Acetylsalicylic acid
Insulin, metformin and glucose reducing sulphonamides
Phenobarbital, phenyloine, primidon, riphampycine;

### 3. Due to Increased Risk of Haemorrhages:

Oral anticoagulants and heparin;

### 4. Other Combinations to be Avoided are:

Antihypertensive agents
Alpha interferon
Weakened ‘live’ vaccines

The present invention is by no means restricted to the compounds, embodiment(s) and methods described by way of example and represented in the accompanying drawings; on the contrary, such methods, compounds and embodiments can be realised according to different variants while still remaining within the scope of the invention.

1. Liposomal gel which, apart from liposomes and a continuous phase, comprises at least one thickening agent, at least one biologically active material and, optionally, at least one additive, whereby the biologically active material is encapsulated in the liposomes, and whereby this material consists of hydrocortisone, its metabolites or precursors, or a mixture thereof, and is applied on the skin for therapeutic purposes, characterised in that the weight ratio phosphatidylcholine/hydrocortisone amounts to at least 2/1 and/or in that the weight ratio phosphatidylcholine/carbomer hydrogel amounts to at least 20/1.

2. The gel according to claim 1, characterised in that the liposomes in the gel are emulsified or suspended.

3. The gel according to claim 1, characterised in that the continuous phase is a watery phase.

4. The gel according to claim 1, characterised in that the thickening agent consists of gel matrices forming a polymeric three-dimensional structure around the liposomes, such as for example a hydrogel formed by a neutralised carbomer or a hydrophilic polymer.

5. The gel according to claim 1, characterised in that the additives are selected from the group formed of sterols, emollients, vitamins, such as for example ascorbyl palmitate or vitamin D3, nutrients, moisturizers, preservatives, radical absorbers, antioxidants, chelators such as EDTA disodium salt, dyes to recognize the concentration of active ingredients, mixtures of different polyethylene glycols, poloxamers such as for example ABA block copolymers with a large variation in the polyethylene over polypropylene ratio, surfactants such as for example dodecyl sulphate and different salts.

6. The gel according to claim 1, characterised in that liposomes are produced whereby the phosphatidylcholine is replaced by another natural phospholipid or by a substance selected from the group consisting of lecithin, phosphatidylethanolamine, lysolecithin, lyophosphatidylethanolamine, phosphatidylserine, phosphatidylinositol, sphingomyelin, cardiolipin, phosphatidic acid, cerebroside, stearyl amine, dipalmitoylphosphatidylcholine, phosphatylcholine, dioleolyl phosphatidylethanolamine, or any equivalent.

7. The gel according to claim 1, characterised in that the liposomes are produced on the basis of synthetic nonionogenic amphiphiles with an aliphatic saturated chain so as to form what are called niosomes.

8. The gel from claim 1, characterised in that it is used in the production of a pharmaceutical preparation to be used in hormonal replacement therapy, i.e. HRT.

9. Method for implementing HRT (hormone replacement therapy), characterised in that it consists in applying a therapeutically effective amount of the gel according to claim 1 on healthy skin in order to maintain or repair the desired hormonal serum concentration.

10. Method according to claim 9, characterised in that the therapeutically effective amount of hydrocortisone is situated between 0.5 and 50% by weight of the ultimate liposomal gel, better still between 1 and 35%, and even better still between 1 and 15%.

11. Commercial packaging for offering the gel according to claim 1, characterised in that it comprises one or several dose measuring means, for example a metering pump, which make it possible to measure a therapeutically effective amount of the gel for use and so as to make it available, or characterised in that it consists of a capsule or similar container which can be opened before use and which contains a therapeutically effective amount of the gel.

12. The gel according to claim 2, characterised in that the continuous phase is a watery phase.
